(19) United States
(12) Patent Application Publication
(10) Pub. No.: US 2023/0371870 A1
(43) Pub. Date: Nov. 23, 2023

(54) SYSTEM AND METHODS FOR ENHANCEMENT OF MEDITATION TRAINING AND PRACTICE

(71) Applicant: Halare, Inc., Sewickley, PA (US)

(72) Inventor: Anthony C. Warren, Wien (AT)

(21) Appl. No.: 18/031,225

(22) PCT Filed: Oct. 13, 2021
(86) PCT No.: PCT/US2021/054675

§ 371 Date: Apr. 11, 2023

Related U.S. Application Data
(60) Provisional application No. 63/092,036, filed on Oct. 15, 2020.

(51) Int. Cl. A61B 5/16; A61B 5/00; A61B 5/0205

(52) U.S. Cl. CPC A61B 5/16; A61B 5/6898; A61B 5/486; A61B 5/0205

(57) ABSTRACT

A portable smart device designed to be held by a user or worn by the user for determining a quality of a meditation session. The portable smart device including a heart rate sensor configured to detect heart rate of the user and output a heart rate signal, and an electronic computing unit coupled to the heart rate sensor. The electronic computing unit including a processor configured to analyze the heart rate signal of the user during the meditation session to determine two or more physiological parameters including at least one of breathing rate, breathing rate variability, heart rate, heart rate variability and vagal tone, and combine the two or more physiological parameters to determine a meditation session quality index (SQI) that indicates the quality of the meditation session.

### FIELD

The present invention relates to systems and methods for enhancing meditation.

### BRIEF DESCRIPTION OF THE DRAWINGS

The invention is best understood from the following detailed description when read in connection with the accompanying drawings, with like elements having the same reference numerals. When a plurality of similar elements is present, a single reference numeral may be assigned to the plurality of similar elements with a capital letter designation referring to specific elements. Included in the drawings are the following figures:

FIG. 1 is a block diagram of a system for monitoring and assessing meditation sessions in persons according to aspects of the invention.

FIGS. 2A, 2B, 2C and 2D are example devices for monitoring and assessing meditation sessions in accordance with aspects of the invention.

FIG. 3 is an example of a pulse trace detected by a blood flow sensor, according to aspects of the invention.

FIG. 4 is an example of a pulse trace detected by a blood flow sensor, according to aspects of the invention.

FIG. 5 is an example in which information can be presented to a MEDITATOR over a period of time, according to aspects of the invention.

FIG. 6 is a flowchart showing a method in which heart rate (HR) is derived from the PPG data obtained from a sensor or camera, according to aspects of the invention.

FIG. 7 is a flowchart showing a method in which heart rate variability (HRV) is derived from the PPG data obtained from a sensor or camera, according to aspects of the invention.

FIG. 8 is a flowchart showing a method in which breathing rate (BR) is derived from the PPG data obtained from a sensor or camera, according to aspects of the invention.

FIG. 9 is a flowchart showing a method in which vagal tone (VT) is derived from the PPG data obtained from a sensor or camera, according to aspects of the invention.

FIG. 10 is a flowchart showing a method in which Session Quality Index SQI(B) is derived, according to aspects of the invention.

FIG. 11 is a flowchart showing a second method in which Session Quality Index SQI(A) is derived, according to aspects of the invention.

FIG. 12 is a flowchart showing how two Session Quality Indices SQI(A) and SQI(B) are used to improve meditation practice, according to aspects of the invention.

### SUMMARY

A portable smart device designed to be held by a user or worn by the user for determining a quality of a meditation session. The portable smart device including a heart rate sensor configured to detect heart rate of the user and output a heart rate signal, and an electronic computing unit coupled to the heart rate sensor. The electronic computing unit including a processor configured to analyze the heart rate signal of the user during the meditation session to determine two or more physiological parameters including at least one of breathing rate, breathing rate variability, heart rate, heart rate variability and vagal tone, and combine the two or more physiological parameters to determine a meditation session quality index (SQI) that indicates the quality of the meditation session.

A method for determining quality of a meditation session. The method is implemented by a portable smart device designed to be held by a user or worn by the user. The method includes detecting, by a heart rate sensor, a heart rate of the user and outputting a heart rate signal, analyzing, by a processor of the portable smart device, the heart rate signal of the user during the meditation session to determine two or more physiological parameters including at least one of breathing rate, breathing rate variability, heart rate, heart rate variability and vagal tone, and combining, by the processor of the portable smart device, the two or more physiological parameters to determine a meditation session quality index (SQI) that indicates the quality of the meditation session.

### BACKGROUND

There is significant interest to improve the general health, quality of life and mental state of the population by training and practicing meditation and mindfulness techniques. In order to bring the benefits of meditation to a large population, a number of software applications using mobile computing devices are available that purport to train and guide novices in the techniques of meditation and the related techniques of mindfulness. Examples include software applications delivered via mobile computing devices from such vendors as Headspace, Calm, Waking Up and others. Meditation training delivered via a mobile device without direct one-to-one instructions, personal feedback and coaching cannot realistically replace training and guidance delivered personally by an expert in one-on-one interactions such as are experienced at so-called meditation retreats or over many hours of expert guidance.

Without direct one-to-one personal instructions and guidance, trainee MEDITATORS may not be aware whether they are performing their meditation SESSIONS correctly, not know how to improve and optimize their meditation techniques, and not know whether their meditation SESSIONS are improving their overall health and quality of life over extended periods.

The systems and methods disclosed herein relate to aiding trainee MEDITATORS wishing to learn the techniques of meditation, in order to: a) monitor the quality of their meditation SESSIONS at a place of their choice without the need for a specialized face-to-face trainer, and/or b) track the benefits of meditation practice over time, and/or c) improve their meditation techniques employing monitored breath training methods.

### DETAILED DESCRIPTION

A novice trainee MEDITATOR usually has difficulty in creating a state of meditation or mindfulness which requires suppressing all thoughts not associated with the present out of their mind. Thus, in such cases, a meditation SESSION may vary between awareness of the past and future, and a focus on only what is happening at that time. As the trainee MEDITATOR shifts between these two states, physiological parameters vary. For example heart rate HR may increase during stages of awareness of the past and future from the HR value in a mindfulness state. The ability to establish a continuous meditative or mindfulness state presents a major barrier in continuing training, causing many trainees to give up at an early stage in their training.

The methods and devices described herein provide a quantitative measure of progress during the early training stages by providing the trainee MEDITATOR (i.e. user of the meditation training system) with a SESSION QUALITY INDEX (SQI) derived from the time intervals when the trainee is in a mindfulness state and an aroused state during a SESSION. We refer to this index as SQI(A). A trainee therefore is able to numerically track their training progress as they learn to establish a mindfulness state for longer periods during SESSIONS. The ability to quantify and track SQI(A) over time provides encouragement to persevere with the training and to learn which techniques are best for them to extend their times in a mindfulness or meditative state.

As a trainee learns to extend their mindfulness or meditative state for longer times during a SESSION, the overall benefit of a SESSION becomes important to quantify. In such cases, the quality of a SESSION depends more on the physiological changes that occur from the start to the end of a SESSION. Two or more physiological parameters are determined at the start and end of a SESSION to create a second SESSION QUALITY INDEX. We refer to this index as SQI(B). The ability to quantify and track SQI(B) over time provides encouragement to persevere with meditation SESSIONS and to learn which techniques are best for the trainee to gain the greatest benefit from a SESSION.

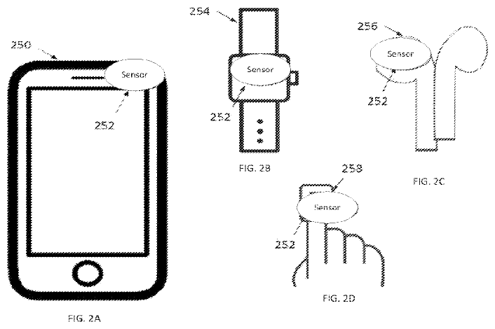

Without a means to quantify and monitor the quality and outcomes of individual meditation SESSIONS and the longer-term benefits derived from multiple SESSIONS, trainee MEDITATORS and even accomplished MEDITATORS may not derive the full benefit from their meditation SESSIONS, or even lose enthusiasm for continuing their training and meditation practice. Means are therefore beneficial to quantify SESSION quality, measure and track the beneficial outcomes of one or more meditation SESSIONS in order to improve meditation training, and meditation practice. If meditation is not achieving targeted goals, means are required to improve meditation practice through monitored breath training. Breath training may include a device (see FIGS. 2A-2D) that instructs the user to control their breath in a certain manner (e.g. hold breath for certain period time, breath for a certain period of time, breath in a rhythmic pattern, etc.), monitors at last one of heart rate, heart rate variability, breathing rate, vagal tone and blood oxygen saturation, and then determines lengths and intensities of occurrences of events such as increased heart rate, increased heart rate variability, increases breathing rate, increased vagal tone, and hypoxic events where the user's blood oxygen level drops below a threshold (e.g. level of adequate oxygen in blood) for a period of time. The device then trains the user to reduce the lengths and the intensities of future events. These occurrences can be monitored by various sensors including but not limited to a heart rate sensor, and blood oxygen saturation sensor (e.g., pulse oximeter).

In one form of meditation referred to as mindfulness, focusing on an “object” that is present in real-time is used to divert consciousness away from thoughts related to past events or in anticipation of future events. In many meditation and mindfulness practices, focusing the mind on breathing is a key technique used to develop meditation skills. Breathing is a partially autonomous and necessary human function that is always available to a MEDITATOR. Therefore, it is one of the most common and important objects employed during meditation sessions and training thereof. Moreover, breathing rhythms are coupled closely with the cardio-vascular system and the two opposing modes of the central nervous system, namely the parasympathetic and sympathetic branches. One of the principal benefits of meditation is the calming of the central nervous system, reducing the sympathetic influence while increasing the parasympathetic influence. The relationship between these two influences are sometimes referred to as vagal tone. This term will be used throughout this application to refer to the balance between the two influences.

Breathing patterns can be used to determine levels of relaxation and anxiety, and the overall balance of the central nervous system. Breathing patterns are also coupled to variations in heart rate, as evidenced in so-called heart rate variability, or HRV. Therefore, monitoring breathing patterns during and subsequent to a meditation session can provide valuable feed-back after a session as well as monitoring short and longer term beneficial outcomes. Such quantification of benefits and progress can in turn, provide encouragement to MEDITATORS who may have reduced their training efforts as a result of lack of quantifiable data on beneficial outcomes. As training can take many months or even years, maintaining motivation and a persistent regimen is important.

Meditation using breathing as an object is more likely to be beneficial if the MEDITATOR has good control over their breathing patterns. Unfortunately, many adults have developed poor breathing behaviors. Such habits as irregular and interrupted breathing rhythms, poor use of the diaphragm, emphasis on oral rather than nasal breathing, sighing and yawning and other breathing disturbances detract from the benefit of using breathing as a meditation object. Therefore, training that combines exercises to improve breathing habits with meditation practice may improve the outcomes of meditation and mindfulness activities.

Meditation is a very personal experience, and ideally training should be provided to meet the specific needs of each MEDITATOR. Further, every meditation session is different; sometimes pure mindfulness states are attained easily, at other times it is difficult to maintain such states. This variability during and between meditation sessions may hinder those training in meditation techniques from gaining full benefit from their training. The techniques described herein provide methods that: a) combine breath improvement training with meditation training, b) quantify the quality of individual training SESSIONS so that a MEDITATOR can confirm their perception of the quality of a meditation session and thereby help to improve techniques for future sessions, and c) track the benefits from meditation sessions over extended times of days, weeks and months.

In order to provide a MEDITATOR with one or more numerical values that quantify the quality of a session, physiological data monitored and recorded during a SESSION are used to create easy to understand indices.

A MEDITATOR's physiological parameters are monitored during breath training and meditation sessions or between sessions using one or more sensors embodied in a wearable computing device such as, but not limited to a watch, arm-band, finger clip, chest-band, ear-clip and insertable ear-bud.

Alternatively, a camera, typically resident in a mobile computing devices such as a so-called smart phone, is placed in contact with or in proximity to a body-part, during breath training and meditation sessions or between sessions.

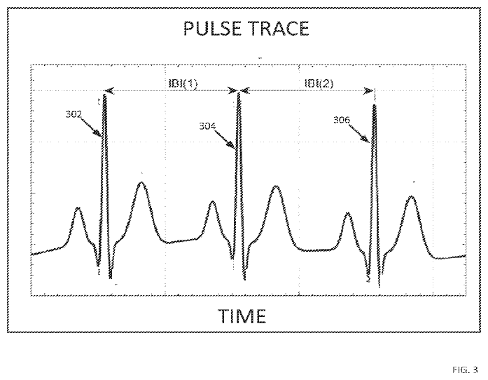

The camera or sensors detect the blood flow in the body part sampling the relative value of flow at a given sampling rate (e.g., at least ten times a second). One form of this type of blood flow data is commonly referred as a photoplethysmograph (PPG). An example of a pulse trace detected by a blood flow sensor is shown in FIG. 3.

The monitored and stored physiological data are used to create one or more “meditation session quality indices (INDICES)”. Examples of such indices include:
a) The percentage of time during the session in which the breathing rate is above or below a pre-determined value.
b) The percentage of time during the session in which the heart rate (HR) is above or below a pre-determined value.
c) The percentage of time during the session in which the heart rate variability (HRV) is above or below a pre-determined value.
d) The percentage of time during the session in which the breathing rate variability is above or below a pre-determined value.
e) The average value of vagal tone (VT) during part or all of the SESSION.
f) The percentage of time during the session in which two or more physiological parameters are below or above their pre-determined values. This combination index is referred to as SQI(A) and is indicative of the time the trainee maintains the mindfulness state during the session.
g) The change in HR between the start and end of meditation SESSION.
h) The change in HRV between the start and end of a meditation SESSION.
i) The change in breathing rate between the start and the end of a meditation SESSION.
j) The change in VT between the start and the end of a meditation SESSION.
k) Combinations of two or more of f)-j) above. This combination index is referred to as SQI(B) and is indicative of the benefit the trainee obtains as a result of the session.

Other INDICES may be calculated from one or more physiological parameters, such as breathing and heart rate.

In addition, or alternatively, the trainee or established MEDITATOR may use the techniques described above to measure physiological parameters and derived indices shortly after a SESSION or at any time thereafter to determine the benefits or otherwise of meditation sessions.

In addition, the trainee MEDITATOR may use the techniques described above to improve their breathing behavior, such improved behavior being supportive in subsequent meditation SESSIONS.

In general, a computing device analyzes and stores the captured data for later display, and the recorded blood flow data is used to determine one or more physiological parameters including but not limited to breathing rate, breathing rate variability, heart rate, heart rate variability, vagal tone, breath holding time, blood oxygen level.

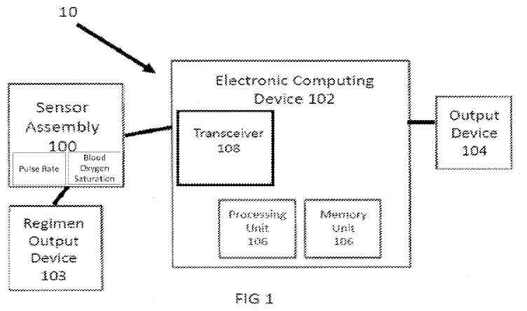

Referring to FIG. 1, a block diagram of a system 10 is shown for capturing the physiological parameters, analyzing the physiological parameters, and determining mediation session quality. The system 10 includes a sensor assembly 100, an electronic computing device 102, and an output device 104 (e.g., display screen, speaker, etc.). Although the sensor assembly 100, the electronic computing device 102, and output device 104 are depicted as separate components in system 10, it is contemplated that any or all of these components may be integrated together in two or one device. For example, the sensor assembly, the electronic computing device 102, and the output device 104 may be integrated into an apparatus attachable to a user (e.g., a wristband, a neckband, other attachments, etc.), or in a smart device, such as a smart phone, tablet computer, laptop computer, etc. Examples of such devices are shown in FIG. 2A as smartphone 250 with a camera and light source acting as a sensor 252 (e.g. heart rate sensor and/or pulse oximeter), FIG. 2B as smartwatch 254 with camera and light source or electrodes acting as a sensor 252 (e.g. heart rate sensor and/or pulse oximeter), FIG. 2C as earbuds 256 with camera and light source or electrodes acting as a sensor 252 (e.g. heart rate sensor and/or pulse oximeter) and FIG. 2D as a finger clamped device 258 (e.g. heart rate sensor and/or pulse oximeter). Other suitable sensors for detecting physiological data of a user will be understood by one of ordinary skill in the art from the description herein.

As referred to herein, a “user” is a person or persons or other entity practicing meditation where the user may receive instructions from the device for performing medication, and breath training sessions where the user may receive instructions from the device for controlling their breathing.

Referring back to FIG. 1, the system 10 includes a regimen output device 103. The regimen output device 103 is adapted to output instructions to the user according to the meditation sessions and breath training sessions established for the user to detect physiological data of the user. The regimen output device 103 may be coupled to the sensor assembly 100, or may be a separate component, such as a smart phone, tablet computer, laptop computer, or other communication device capable of providing breath training instructions to the user.

The system 10 further includes an electronic computing device 102 with a processing unit 106 (e.g., including a processor), a transceiver 108, and a memory unit 110. The transceiver 108 may be utilized to receive physiological data detected from the sensor assembly 100. In embodiments where the electronic computing device 102 is integrated with the sensor assembly 100, the transceiver 108 may not be a necessary component for the transmission and reception of data to be analyzed by the electronic computing device 102. The memory unit 110 is depicted as integrated into the electronic computing device 102. It is contemplated that additional memory units may be utilized, such as a memory unit integrated into the sensor assembly 100 or a cloud storage device. Such memory units are configured to store detected physiological data and subsequent analyzed data.

The processing unit 106 is adapted to process the data detected by the sensor assembly 100 according to particular algorithms to determine a quality of meditation sessions. The particular algorithms the processing unit 106 applies to the physiological data detected by the sensor assembly 100 depend upon the type of data detected and the sensors that are used to detect the data. Although the algorithms described herein are related to an individual sensor type, the physiological data analyzed from each type of sensor may be used in conjunction with or in combination with data from other sensors to determine meditation session quality.

As one example of how a physiological parameter can be derived from the PPG data obtained from a sensor or camera, heart rate is calculated by detecting the peaks 302, 304 and 306 of each pulse cycle as illustrated in FIG. 3 and counting such peaks over a defined time period, (e.g., 1 minute). The number of peaks in that time is the heartrate in beats per minute. Allowances are made to detect missed or ectopic beats, or longer periods of arrhythmia. In practice, the heartrate is calculated over multiple seconds (e.g., 10 seconds) with the window of calculation advancing one second for each subsequent second. For example, a heart rate value would be counted for session times 0-10, 1-11, 2-12, etc., so that heartrate can be tracked as the MEDITATOR undertakes a session. Of particular interest is the heart rate (HR) detected at the start of a SESSION and that at the end of the same SESSION. Assuming a SESSION lasts for n seconds, the start and end values will be derived in windows 0-10 seconds and n-10 seconds respectively. These two values are referred to as HR(START) and HR(END). In practice HR(START) may be derived from a later time window to allow for the MEDITATOR settle down, but the delay should not be more than a given amount (e.g., window of 50-60 seconds). A gradual diminution of heartrate during a SESSION would indicate effective MEDITATION.

As a second example of how a physiological parameter can be derived from the PPG data obtained from a sensor or camera, heart rate variability (HRV) is calculated by detecting the peak of each pulse cycle and calculating an Interbeat Interval (IBI), as illustrated in FIG. 3 as IBI(1) and IBI(2), between two subsequent peaks (e.g., between 302 and 304, and between peaks 304 and 306) over a defined time period (e.g., 1 minute). Allowances are made to detect missed or ectopic beats, or longer periods of arrhythmia. In practice, HRV is calculated over multiple seconds (e.g., 10 seconds) with the window of calculation advancing one second for each subsequent second. For example, an HRV value would be counted for session times 0-10, 1-11, 2-12, etc., so that HRV can be tracked as the MEDITATOR undertakes a session. Of particular interest is HRV detected at the start of a SESSION and that at the end of the same SESSION. Assuming a SESSION lasts for n seconds, the start and end values will be derived in windows 0-10 seconds and n-10 seconds respectively. These two values are referred to as HRV(START) and HRV(END). In practice HRV(START) may be derived from a later time window to allow for the MEDITATOR settle down, but the delay should not be more than a given amount of time (e.g., window of 50-60 seconds). A gradual increase of HRV during a session would indicate effective MEDITATION.

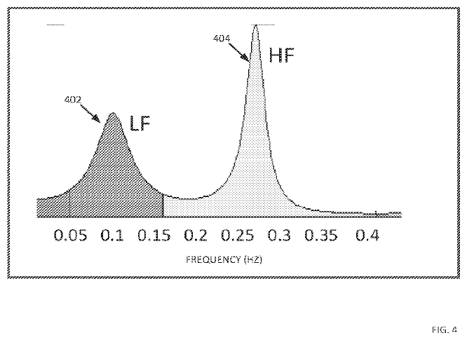

As a third example of how a physiological parameter can be derived from the PPG data obtained from a sensor or camera, breathing rate (BR) is calculated by detecting the dominant frequency that heartrate varies with time. Heartrate varies in synchrony with breathing. One method of calculating BR therefore is to perform a frequency analysis of the heartrate and determine the most dominant frequency component using techniques such as Fourier transformation and analysis. FIG. 4 shows a typical spectrum of HRV derived from a series of IBP s, n), where n is greater or equal to 100. The greater the value of n, the more accurate the frequency spectrum is. In practice a value of n of 300 or greater is preferred. In practice breathing frequencies lie in a range between 9 and 24 breaths a minute. This can be expressed as a range of frequencies 0.15 to 0.4 Hertz. This range is labeled as HF 404 in FIG. 4. Therefore, the computing algorithms employ a bandpass filter to eliminate frequencies outside that range when determining breathing rate and detect the peak frequency in the HF range. As one example, in FIG. 4, this peak frequency is 0.275 Hertz which is equivalent to a breathing rate of 16.5 breaths a minute. Allowances are made to detect missed or ectopic beats, or longer periods of arrhythmia. In practice, breathing rate is calculated for multiple minutes (e.g., 3 minutes) with the window of calculation advancing one minute for each subsequent minute. For example, a breathing rate value would be counted for session times 0-3,1-4, 2-5, 3-6 minutes, etc., so that breathing rate can be tracked as the MEDITATOR undertakes a session. Of particular interest is breathing rate (BR) detected at the start of a SESSION and that at the end of the same SESSION that is in time windows, 0-300 and n-300 to n, where n is the SESSION end time. These two values are referred to as BR(START) and BR(END). In practice HBRSTART) may be derived from a later time window to allow for the MEDITATOR settle down, but the delay should not be more than a given amount of time (e.g. window of 60-360 seconds). A gradual decrease of breathing rate during a session would indicate effective MEDITATION.

As a fourth example of how a physiological parameter can be derived from the PPG data obtained from a sensor or camera, vagal tone (VT) is calculated by converting the time sequence of pulse peaks to the frequency domain using methods such as Fourier Transformation analysis. Algorithms employ two bandpass filters (e.g. 0.15 to 0.4 Hertz and 0.05 to 0.15 Hertz). These two frequency bands are defined as HF 404 and LF 402 as illustrated in FIG. 4. Integration algorithms calculate the areas under the HF and LF bands INT(HF) and INT(LF) respectively. The ratio INT(HF)/INT(LF) is then calculated. This ratio represents a value of for VT. INT(HF) is a measure of the energy expressed by the sympathetic branch of the central nervous system, and INT(HF) is a measure of the energy expressed by the parasympathetic branch of the central nervous system. A ratio of the two is indicative of a central nervous system in balance. Allowances are made to detect missed beats, or longer periods of arrhythmia. In practice, VT is calculated for a period of time (e.g., 10 minutes) with the window of calculation advancing one minute for each subsequent minute. So, for example a VT value would be counted for session times 0-10,1-11, 2-12, 3-13 minutes, etc., so that VT can be tracked as the MEDITATOR undertakes a session. Of particular interest is VT detected at the start of a SESSION and that at the end of the same SESSION, that is in time windows, 0-600 and n-600 to n, where n is the SESSION end time. These two values are referred to as VT(START) and VT(END). In practice, (VTSTART) may be derived from a later time window to allow for the MEDITATOR to settle down, but the delay should not be more than a given time period (e.g., window of 60-720 seconds). A gradual decrease of vagal tone during a session would indicate effective MEDITATION. Such derived physiological data described in paragraphs above may be combined to create SESSION QUALITY INDICES (SQI's) (e.g. SQI(A) and SQI(B)) in order to provide MEDITATORS simple to understand information to help them: a) quantify the effectiveness of a single SESSION, b) compare one or more SESSIONS, c) track the effectiveness of MEDITATION training and practice over time, and d) determine whether monitored breath training should be employed to improve meditation techniques.

One index that measures the decline in HR during a SESSION. Heartrate is measured for a period (e.g. one minute) at the start of session and again over a period say the last minute of a session as described above. The percentage change per minute of the SESSION is calculated and recorded as ΔHR.

A second index that measures the increase in HRV during a SESSION. HRV is measured for a period, say one minute at the start of session and again over a period say the last minute of a session as described above. The percentage change per minute of the SESSION is calculated and recorded as ΔHRV.

A third index measures the decrease in breathing rate during a SESSION. Breathing rate is measured for a period (e.g. one minute) at the start of session and again over a period say the last minute of a session as described above. The percentage change per minute of the SESSION is calculated and recorded as ΔBR.

A fourth index that measures the decrease in vagal tone during a SESSION. Vagal Tone is measured for a period (e.g. ten minutes) at the start of session and again over a period (e.g. the last ten minutes) of a session as above. The percentage change per minute of the SESSION is calculated and recorded as ΔVT.

Indices may also be used in combination.

In one example, a SESSION quality index SQI(A) is computed by summing the indices each with a weighting factor WF and the % Time below the predetermined thresholds as follows.

SQI(A)=WF(HR)*% TimeHR+WF(HRV)*% TimeHRV+WF(BR)*% TimeBR+WF(VT)*% TimeVT.

Weighting factors total to a scaled value (e.g. 1), and are determined depending on the individual values of % Time. An increase in SQI(A) indicates an increase in the mindfulness state during the session. A decrease in SQI(A) indicates a decrease in the mindfulness state during the session. Table 1 below illustrates an example of how WF's are calculated:

TABLE 1

| | % Time Below | |
|---|---|---|
| | Thresholds | WF's |
| % TimeHR | 50 | 0.3125 |
| % TimeHRV | 20 | 0.125 |
| % TimeBR | 60 | 0.375 |
| % TimeVT | 30 | 0.1875 |
| Total | | 1 |

In another example, a SESSION quality index SQI(B) is computed by summing the indices each with a weighting factor WE as follows.

SQI(B)=WF(HR)*ΔHR+WF(HRV)*ΔHRV+WF(BR)*ΔBR+WF(VT)*ΔVT.

Weighting factors total to a scaled value (e.g., 1), and are determined depending on the individual values of each A value in percentages. An increase in SQI(B) indicates an increase in benefit of the session. A decrease in SQI(B) indicates a decrease in the benefit of the session. Table 2 below illustrates an example of how WF's are calculated:

TABLE 2

| | % Change | WF's |
|---|---|---|
| ΔHR | 50 | 0.3125 |
| ΔHRV | 20 | 0.125 |
| ΔBR | 60 | 0.375 |
| ΔVT | 30 | 0.1875 |
| Total | | 1 |

If one or more of the changes cannot be determined, the calculation is adjusted to compensate. Table 2 shows the case where ΔVT is not determined. If one or more other of ΔHR, ΔHRV, ΔBR, ΔVT cannot be determined, weighting factors will be calculated based on those that are determined. For example, if only ΔHR is known, then it will have a weighting factor of 1. Table 3 below shows an example where ΔVT cannot be determined and is therefore assigned a weight factor of 0 and the remaining weighting factors are adjusted to compensate:

TABLE 3

| | % Change | WF's |
|---|---|---|
| ΔHR | 50 | 0.384615 |
| ΔHRV | 20 | 0.153846 |
| ΔBR | 60 | 0.461538 |
| ΔVT | | 0 |
| Total | | 1 |

MEDITATORS may have difficulty in maintaining a meditative or mindfulness state throughout a SESSION. In such cases it is useful to monitor variations of certain physiological parameters to determine how often and for how long a MEDITATOR deviates from such states For example, the length of time that the breathing rate is below a pre-determined value during a session may be one such index, or the length of time that heart rate variability exceeds a pre-determined value may be used to create another index, or another index determined from the balance between the parasympathetic and sympathetic branches of the central nervous system, the vagal tone, may be used. Pre-determined values may be defined using a database of values derived all or in part from a population of comparable phenotypes to the MEDITATOR. A SESSION QUALITY INDEX, SQI(A) of particular value to a novice TRAINEE is derived from determining the percentage of time two or more physiological parameters exceed or are less than pre-determined values. SQI(A) helps the trainee increase their mindfulness or meditative states during a SESSION by identifying external influences that detract from these states, learning the techniques that enhance these states, and tracking over time the improvements in SQI(A) to improve long term adherence to SESSIONs. For example, if HR exceeds the HR predetermined value for 50% of the session, this will affect SQI(A) indicating that the trainee may have only been in a mindfulness state for 50% of the session.

A second SESSION QUALITY INDEX, SQI(B) of particular value to a MEDITATOR is derived from determining the changes in two or more physiological parameters from the start to the completion of a SESSION. SQI(B) helps the MEDITATOR identify external influences that detract from the benefit from a SESSION, learning the techniques that enhance meditation states, and tracking over time the improvements in SQI(B) to improve long term adherence to SESSIONs. For example, if during the start of the sessions, HR has a value of 100 beats per minute, and at the end of the session, HR has a value of 80 beats per minute, this will affect SQI(B) indicating that the trainee has obtained some benefit of the session because their HR decreased over the course of the session.

Parameters may also be used in combination. For example, an SQI is created from the time interval when both low breathing rate and high heart rate variation occur simultaneously. Other useful indices based on breathing and heart rate metrics could also be utilized.

Bio-feedback during meditation sessions has been used as aid to develop slow, relaxed and regular breathing patterns. The methods/devices described herein can be implemented both with and without real-time bio-feedback methods to develop desirable breathing patterns. The methods/devices herein, monitor and record physiological parameters which may be related to breathing behavior and these may be used to guide breathing behavior during a meditation session. Alternative the MEDITATOR may be restricted from being made aware of the parameters in real-time during a meditation session.

The systems and methods disclosed herein relate to aiding trainee MEDITATORS wishing to learn the techniques of meditation, in order to: a) improve their breathing behavior, and/or b) monitor the quality of their meditation sessions at a place of their choice without the need for a specialized face-to-face trainer, and/or c) additionally track the benefits of meditation practice over time.

Additionally, the systems and methods disclosed herein relate to aiding established MEDITATORS wishing to further develop the techniques of meditation, in order to: a) improve their breathing behavior, and/or b) monitor the quality of their meditation sessions at a place of their choice without the need for a specialized face-to-face trainer, and/or c) additionally track the benefits of meditation practice over time.

During part or all of a meditation session (SESSION), one or more sensors monitor one or more physiological parameters including but not limited to breathing rate, breathing rate variability, heart rate, heart rate variability, vagal tone, breath holding time and blood oxygen level.

After a SESSION, which may be guided or self-imposed, the MEDITATOR may access the physiological data recorded and stored during the SESSION and/or the SQI or indices derived from such physiological data. The session data enable the MEDITATOR to match their personal impression of the quality of the session with actual quantified measurements of the physiological parameters or indices derived from such parameters that occurred during the SESSION. Such comparison helps the MEDITATOR to comprehend what they did in the SESSION that was helpful or what was deleterious and how they might improve their techniques for later SESSIONS.

Moreover, changes in physiological data and derived indices from subsequent SESSIONS can be used to determine trends over time of benefits from meditation SESSIONS.

A MEDITATOR may also determine the values of their physiological data outside a SESSION to determine overall health benefits that may derive from SESSIONS over time.

Additionally, a meditation trainee may use physiological data detected by sensors to guide improvements in breathing behavior during a meditation session or in a separate session devoted to solely improving breathing behavior as described above.

Specifically, if the MEDITATOR is unable to achieve values of one of more of ΔHR, ΔHRV, ΔBR, ΔVT (or % TimeHR, % TimeHRV, % TimeBR, % TimeVT) during one or more SESSIONS exceeding defined thresholds, the MEDITATOR may undertake personalized breath training using the sensor devices shown in FIGS. 2A-2D to monitor physiological parameters during the breath training.

Threshold values may be defined with reference to a database which matches a MEDITATOR to thresholds determined from matching phenotypes. Or fixed thresholds may be used according to an example in Table 4 below:

TABLE 4

| | % Threshold |
|---|---|
| ΔHR | <0 |
| ΔHRV | <0 |
| ΔBR | <2 |
| ΔVT | <5 |

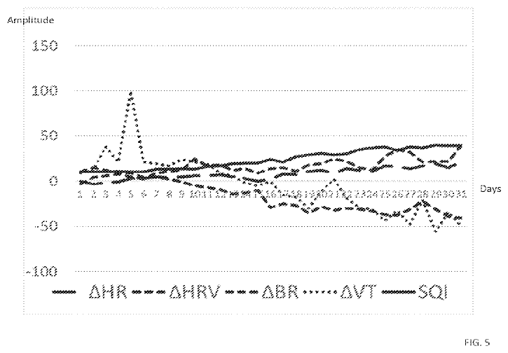

Similar thresholds could be used based on % TimeHR, % TimeHRV, % TimeBR and % TimeVT. Additional physiological parameters that may be monitored in activities referenced include but are not limited to breath holding times, blood oxygen level etc. Subsequent to a SESSION, the MEDITATOR may access the physiological data and SQI's which quantify the quality of the SESSION and/or the longer term trends of multiple SESSIONS. Such analysis enables the MEDITATOR to compare the perceived quality of the SESSION with the impact the session had on physiological parameters and INDICES known to correlate with SESSION quality. FIG. 5 illustrates one way in which the information can be presented to a MEDITATOR over a period of one month. Other means of presentation include but are not limited to, bar charts, pie charts, tables of data, scrolling charts and line diagrams. Access to individual SESSION data and longer term trends in both individual changes in physiological parameters and an aggregated SQI can: a) indicate to the MEDITATOR which SESSIONS are less or more effective, b) indicate whether physiological parameters known to relate to improved wellness and health are being improved from meditation Sessions, c) determine whether the MEDITATOR should undertake monitored breathing exercises to improve meditation techniques

Specifically, if the MEDITATOR is unable to achieve values of one of more of ΔHR, ΔHRV, ΔBR, ΔVT during one or more SESSIONS exceeding defined thresholds, the MEDITATOR may undertake personalized breath training using the sensor devices shown in FIGS. 2A-2D to monitor physiological parameters during such training as described above.

Threshold values may be defined with reference to a database which matches a MEDITATOR to a thresholds determined from matching phenotypes. Or fixed thresholds may be used according to Table 5 below:

TABLE 5

| | Threshold |
|---|---|
| ΔHR | <0 |
| ΔHRV | <0 |
| ΔBR | <2 |
| ΔVT | <5 |

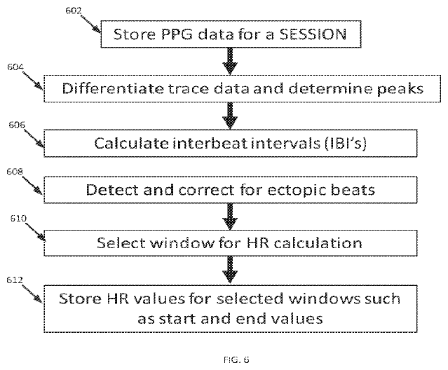

FIG. 6 is a flowchart showing a method in which heart rate (HR) is derived from the PPG data obtained from a sensor or camera. In this example, the processor stores the PPG data in step 602, differentiates the PPG data and then uses the differentiated PPG data to determine peaks in the pulse trace in step 604. The processor then calculates the interbeat intervals, IBIs in step 606, detects and corrects for ectopic beats in step 608, for example by inserting a missing beat based on juxtapositioned IBI's, selects a window for HR calculation in step 610 and stores HR values for the selected window such as start HR values and end HR values in step 612. Start HR values may be delayed from start of a SESSION to allow MEDITATOR to settle into meditation.

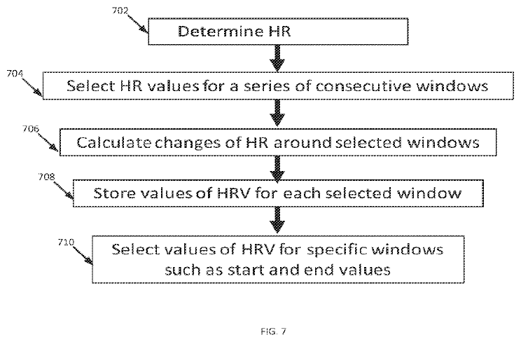

FIG. 7 is a flowchart showing a method in which heart rate variability (HRV) is derived from the PPG data obtained from a sensor or camera. In this example, the processor determines HR in step 702 as described with respect to FIG. 6, selects HR values in step 704 for a series of consecutive windows, calculates changes of HR around the selected windows in step 706 and stores the changes of HR as the HRV values for each window in step 708. The processor then selects values of HRV in step 710 for windows such as start HRV values and end HRV values. Start HRV values may be delayed from start of a SESSION to allow MEDITATOR to settle into meditation.

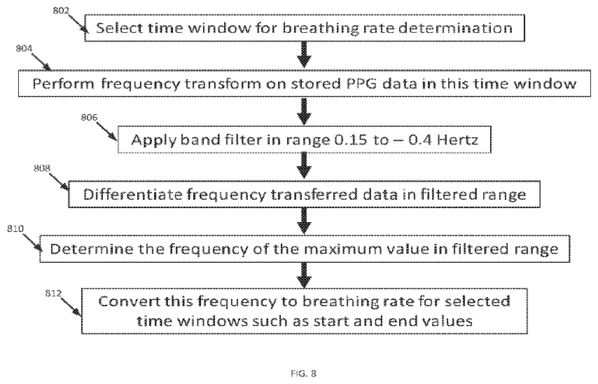

FIG. 8 is a flowchart showing a method in which breathing rate (BR) is derived from the PPG data obtained from a sensor or camera. In this example, the processor selects a time window in step 802 for breathing rate determination, performs a frequency transform (e.g., a Fast Fourier Transform, FFT) in step 804 on the stored PPG data in this window, and applies a bandpass filter (e.g. 0.15 Hz to 0.4 Hz) in step 806 to select the High Frequency (HF) section of the frequency spectrum. The processor then differentiates in step 808 the filtered data and determines in step 810 the frequency of the maximum value in the filtered range. The processor then converts this frequency to breathing rate in step 812 for selected time windows such as start BR values and end BR values. Start BR values may be delayed from start of a SESSION to allow MEDITATOR to settle into meditation. Time windows selected for BR determination are usually longer than those for HR and HRV.

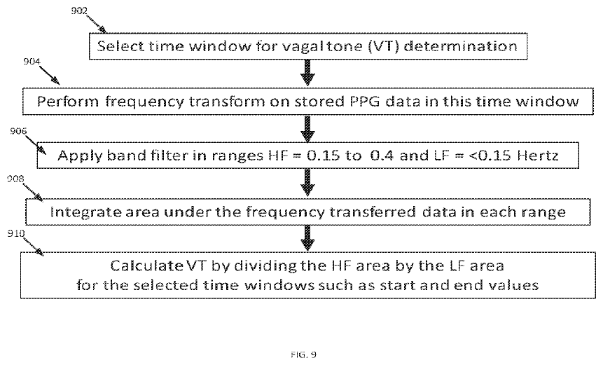

FIG. 9 is a flowchart showing a method in which vagal tone (VT) is derived from the PPG data obtained from a sensor or camera. In this example, the processor selects a tie window for VT determination in step 902 and performs a frequency transform (e.g. FFT) on stored PPG data in this time window in step 904. The processor then applies a bandpass filters (e.g. HF=0.15 Hz to 0.4 Hz and LF=0 Hz to 0.15 Hz) in step 906 and integrates the filtered data in step 908. The processor computes VT in step 910 by dividing the HF area by the LF area for the selected time windows such as start VT values and end VT values. Start VT values may be delayed from start of a SESSION to allow MEDITATOR to settle into meditation. Time windows selected for VT determination are usually longer than those for HR and HRV.

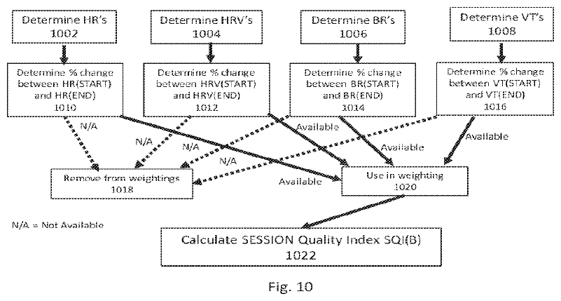

FIG. 10 is a flowchart showing a method in which SQI(B) is derived. In this example, the processor determines one or more of HR in step 1002, HRV in step 1004, BR in step 1006 and VT in step 1008 as described in FIGS. 6-9. The processor determines percent change of each of HR in step 1010, HRV in step 1012, BR in step 1014 and VT in step 1016 between the start and end of the window (e.g., percent change between HR(start) and HR(end), etc.). When available, HR, HRV, BR and/or VT are weighted accordingly and combined to compute the SQI(B). When not available, HR, HRV, BR and/or VT are removed from the weightings (e.g. given a weight of zero) in steps 1018 and 1020. Start time windows selected for one or more physiological parameters may be delayed to allow MEDITATOR to settle into meditation. In step 1022, the processor computes the SQI(B).

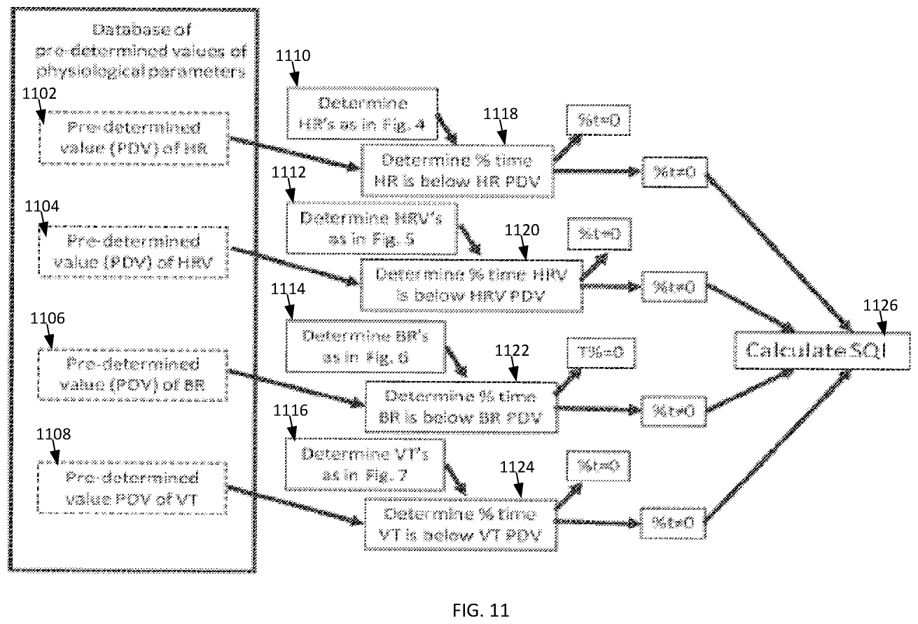

FIG. 11 is a flowchart showing a second method in which SQI(A) is derived. In this example, the processor determines one or more of HR in step 1110, HRV in step 1112, BR in step 1114 and VT in step 1116 as described in FIGS. 6-9. The processor determines the percentage of time within a SESSION that one or more of HR in step 1118, HRV in step 1120, BR in step 1122 and VT in step 1124 exceed (HR, BR, VT) or fall below (HRV) pre-determined values (PDVs) 1102, 1104, 1106 and 1108 stored in a database. PDVs may be fixed in value or derived by comparing the MEDITATOR with similar phenotypes. When available, the percentage times are weighted accordingly and combined to compute the SQI(A) in step 1126. When not available, HR, HRV, BR and/or VT percentage times are removed from the weightings (e.g. given a weight of zero). Start time windows selected for one or more physiological parameters may be delayed to allow MEDITATOR to settle into meditation.

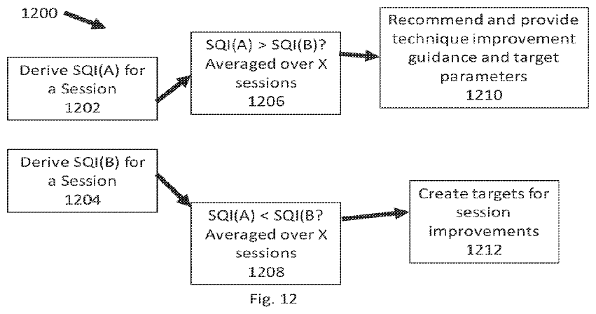

FIG. 12 is a flowchart showing a method 1200 how two specific session quality indices SQI(A) and SQI(B) are used to benefit meditation training and practice. For each session, SQI(A) and SQI(B) are calculated in steps 1202 and 1204, and the differences between them determined. If SQI(A) is greater than SQI (B) over several consecutive sessions X in step 1206, the MEDITATOR has not fully developed the mindfulness techniques required for fully beneficial sessions. In one example, the value of X is generally in the range of 3-7 sessions. In this case, recommendations are made to the trainee in step 1210 on how to improve meditation techniques such as by recommending breath training exercises. Targets for two or more physiological parameters are provided. If SQI(B) is greater than SQI (A) over several consecutive sessions X in step 1208, the MEDITATOR has substantially acquired the mindfulness techniques required for fully beneficial sessions. In this example, the value of X is in the range of 3-7. In this case, targets are set in step 1212 for further improvements to session quality as determined by enhanced values for changes in two or more physiological parameters.

1. A portable smart device designed to be held by a user or worn by the user, the portable smart device for determining a quality of a meditation session, the portable smart device comprising:
a heart rate sensor configured to detect heart rate of the user and output a heart rate signal; and
an electronic computing unit coupled to the heart rate sensor, the electronic computing unit comprising a processor configured to:
analyze the heart rate signal of the user during the meditation session to determine two or more physiological parameters including at least one of breathing rate, breathing rate variability, heart rate, heart rate variability and vagal tone, and combine the two or more physiological parameters to determine a meditation session quality index (SQI) that indicates the quality of the meditation session.

2. The portable smart device of claim 1, wherein the processor is further configured to use changes of the two or more physiological parameters near the start and near the end of a meditation session to determine the meditation SQI.

3. The portable smart device of claim 1, wherein the processor is further configured to weight the two or more physiological parameters according to their individual changes to determine the meditation SQI.

4. The portable smart device of claim 1, wherein the processor is further configured to monitor the two or more physiological parameters after a meditation session ends to determine a meditation SQI.

5. The portable smart device of claim 1, wherein the processor is further configured to provide breath training exercises monitored by the heart rate sensor if the changes in the two or more physiological parameters during a session are below a specified threshold value.

6. The portable smart device of claim 1, wherein the processor is further configured to determine a quality of multiple meditation sessions by monitoring the two or more physiological parameters during the multiple meditation sessions.

7. A method for determining quality of a meditation session, the method implemented by a portable smart device designed to be held by a user or worn by the user, the method including:
detecting, by a heart rate sensor, a heart rate of the user and outputting a heart rate signal;
analyzing, by a processor of the portable smart device, the heart rate signal of the user during the meditation session to determine two or more physiological parameters including at least one of breathing rate, breathing rate variability, heart rate, heart rate variability and vagal tone; and
combining, by the processor of the portable smart device, the two or more physiological parameters to determine a meditation session quality index (SQI) that indicates the quality of the meditation session.

8. The method of claim 7, further comprising: using, by the processor of the portable smart device, changes of the two or more physiological parameters near the start and near the end of a meditation session to determine the meditation SQI.

9. The portable smart device of claim 7, further comprising: weighting, by the processor of the portable smart device, the two or more physiological parameters according to their individual changes to determine the meditation SQI.

10. The portable smart device of claim 7, further comprising: monitoring, by the processor of the portable smart device, the two or more physiological parameters after a meditation session ends to determine a meditation SQI.

11. The portable smart device of claim 7, further comprising: providing, by the processor of the portable smart device, breath training exercises monitored by the heart rate sensor if the changes in the two or more physiological parameters during a session are below a specified threshold value.

12. The portable smart device of claim 7, determining, by the processor of the portable smart device, a quality of multiple meditation sessions by monitoring the two or more physiological parameters during the multiple meditation sessions.
